(19) United States
(12) Patent Application Publication
(10) Pub. No.: US 2024/0225873 A1
(43) Pub. Date: Jul. 11, 2024

(54) ANTI-MIGRATION STENT

(71) Applicant: Boston Scientific Scimed Inc., Maple Grove, MN (US)

(72) Inventors: Yeison Calvo, San Ramón (CR); Gian Franco Loo Fuchs, San Jose (CR); John Thomas Favreau, Shrewsbury, MA (US); Richard Kim, Chestnut Hill, MA (US); Lisa Margaret Sangree, Boston, MA (US); Anthony Frank Tassoni, JR., Andover, MN (US); Serena Scott, Worcester, MA (US); Andrew James Schaubhut, Bolton, MA (US); Robb Morse Gavalis, Westborough, MA (US)

(73) Assignee: Boston Scientific Scimed Inc., Maple Grove, MN (US)

(21) Appl. No.: 18/405,447

(22) Filed: Jan. 5, 2024

Related U.S. Application Data
(60) Provisional application No. 63/437,499, filed on Jan. 6, 2023.

(51) Int. Cl. A61F 5/00

(52) U.S. Cl. CPC A61F 5/0076; A61F 5/0036

(57) ABSTRACT

Stents, auxiliary compressive structure for stents, and techniques to avoid migration of a deployed stent are provided. An auxiliary support structure can be provided to apply compressive force on the flanges of the stent further compressing the stent or to provide axial or radial support for the flanges. The auxiliary support structures can be integrated into the stent or provided as separate device.

### CROSS-REFERENCE TO RELATED APPLICATIONS

This application claims the benefit of U.S. Provisional Patent Application Ser. No. 63/437,499 filed Jan. 6, 2023, the disclosure of which is incorporated herein by reference.

### TECHNICAL FIELD

The present disclosure relates generally to the field of implantable medical devices, and related systems and methods for adjusting accessibility through a passage of a medical device. More particularly, the present disclosure relates to devices, systems, and methods for bridging two anatomical structures, such as a lumen-apposing device.

### BACKGROUND

Treatment methods for various medical conditions, such as obesity, diabetes, pancreatic pseudocysts, biliary obstruction, and duodenal ulcers, involve creating anastomoses between the stomach or duodenum and other structures such as pancreatic fluid collections, the gallbladder, bile ducts, or the jejunum. A lumen-apposing device may be placed between the stomach or duodenum and another organ to allow for passage of materials (fluid, liquid, chyme, food, etc.) between them. One challenge presented by such devices is to prevent migration of the device distally into the other organ or proximally into the stomach or duodenum. Thus, there is an ongoing need to provide alternative medical devices as well as alternative methods for manufacturing and using medical devices.

### BRIEF SUMMARY

This disclosure provides design, material, manufacturing method, and use alternatives for medical devices. An embodiment includes a system, comprising a radially expanding tubular framework having a first end region, a second end region, a medial region positioned between the first end region and the second end region, and a lumen extending from the first end region to the second end region, wherein the first end region comprises a first flange and the second end region comprises a second flange, and an auxiliary support structure configured to provide support to the first flanges and the second flange when the stent is in an expanded state.

Further embodiments can include the system above, wherein the first flange and the second flange are configured to atraumatically engage a bodily tissue.

Further embodiments can include the system above, wherein the auxiliary support structure comprises a second radially expanding tubular framework, the second radially expanding tubular framework having a first end region, a second end region, a medial region positioned between the first end region and the second end region, and a lumen extending from the first end region to the second end region, wherein the first end region of the second radially expanding tubular framework comprises a first flange and the second end region of the second radially expanding tubular framework comprises a second flange.

Further embodiments can include the system above, wherein the first flange and the second flange of the first radially expanding tubular framework have a first diameter and the first flange and the second flange of the second radially expanding tubular framework have a second diameter less than or equal to the first diameter.

Further embodiments can include the system above, wherein the first radially expanding tubular framework has a first longitudinal compressive force and the second radially expanding tubular framework has a second longitudinal compressive force greater than the first longitudinal compressive force.

Further embodiments can include the system above, wherein the auxiliary support structure comprises a proximal flange retention member, a distal flange retention member, and a spring coupled between the proximal and distal flange retention members.

Further embodiments can include the system above, wherein the first flange and the second flange of the radially expanding tubular framework have a first diameter and wherein the proximal and distal flange retention members comprise a second diameter, wherein the second diameter is greater than or equal to the first diameter minus 5 millimeters (mm) and less than or equal to the first diameter plus 5 mm.

Further embodiments can include the system above, wherein the spring has an unstretched length less than or equal to the foreshortened length of the stent.

Further embodiments can include the system above, wherein the auxiliary support structure comprises at least one spring integrally coupled with the first flange and the second flange, the at least one spring biased to apply compressive force upon the flanges when the stent is in the foreshortened state.

Further embodiments can include the system above, wherein the spring comprises pleats or folds biased to apply compressive force on the first flange and the second flange.

Further embodiments can include the system above, wherein the spring comprises coils.

Further embodiments can include the system above, wherein the auxiliary support structure applies a compressive force onto the stent reducing the longitudinal length of the stent and increasing or maintaining the radial diameter of the first flange and the second flange.

Further embodiments can include the system above, wherein the stent comprises a coating.

Further embodiments can include the system above, wherein the stent and/or the auxiliary support structure comprises nitinol.

Further embodiments can include the system above, wherein the radially expanding tubular framework includes a coating applied over the radially expanding tubular framework.

Another embodiment includes a stent comprising: a radially expanding tubular framework having a first end region, a second end region, a medial region positioned between the first end region and the second end region, and a lumen extending from the first end region to the second end region, wherein the first end region comprises a first flange and the second end region comprises a second flange, and at least one spring integrally coupled with the first flange and the second flange, the at least one spring biased to apply compressive force upon the flanges when the stent is in the foreshortened state.

Further embodiments can include the stent above, wherein the spring comprises pleats or folds biased to apply compressive force on the first flange and the second flange.

Further embodiments can include the stent above, wherein the spring comprises coils.

Further embodiments can include the stent above, wherein the auxiliary support structure applies a compressive force onto the stent reducing the longitudinal length of the stent and increasing or maintaining the radial diameter of the first flange and the second flange.

Further embodiments can include the stent above, wherein the stent comprises a coating.

Further embodiments can include the stent above, wherein the stent and/or the auxiliary support structure comprises nitinol.

Further embodiments can include the stent above, wherein the radially expanding tubular framework includes a coating applied over the radially expanding tubular framework.

Another embodiment can include a method comprising: forming an anastomosis in tissue; deploying a radially expanding tubular framework into the anastomosis, the radially expanding tubular framework comprising a first end region, a second end region, a medial region positioned between the first end region and the second end region, and a lumen extending from the first end region to the second end region, wherein the first end region comprises a first flange and the second end region comprises a second flange, and deploying an auxiliary support structure around the deployed radially expanding tubular framework.

Further embodiments can include, wherein the auxiliary support structure comprises a second radially expanding tubular framework, the second radially expanding tubular framework having a first end region, a second end region, a medial region positioned between the first end region and the second end region, and a lumen extending from the first end region to the second end region, and wherein the first end region of the second radially expanding tubular framework comprises a first flange and the second end region of the second radially expanding tubular framework comprises a second flange.

Further embodiments can include, wherein the first flange and the second flange of the first radially expanding tubular framework have a first diameter and the first flange and the second flange of the second radially expanding tubular framework have a second diameter less than or equal to the first diameter.

Further embodiments can include, wherein the first radially expanding tubular framework has a first longitudinal compressive force and the second radially expanding tubular framework has a second longitudinal compressive force greater than the first longitudinal compressive force.

Further embodiments can include, wherein the auxiliary support structure comprises a proximal flange retention member, a distal flange retention member, and a spring coupled between the proximal and distal flange retention members.

The above summary of some embodiments is not intended to describe each disclosed embodiment or every implementation of the present disclosure. The disclosure that follows more particularly exemplifies these embodiments.

### BRIEF DESCRIPTION OF THE DRAWINGS

The disclosure may be more completely understood in consideration of the following detailed description in connection with the accompanying drawings, in which:

FIG. 1 illustrates a perspective view of a stent positioned between a stomach and a portion of a small intestine;

FIG. 2 illustrates a cross-section view of the stent positioned between the stomach and the portion of a small intestine take at line 2-2 of FIG. 1;

FIG. 3A and FIG. 3B illustrate an embodiment of a stent;

FIG. 4 illustrates an embodiment of a stent flange retention member.

FIG. 5 illustrates an embodiment of another stent flange retention member;

FIG. 6 illustrates a stent delivery device;

FIG. 7 illustrates a deployed stent with a retention member;

FIG. 8A and FIG. 8B illustrate a stent and stent deployment device;

FIG. 9A and FIG. 9B illustrate a stent and retention member;

FIG. 10 illustrates an auxiliary compressive support structure for a stent;

FIG. 11A, FIG. 11B, and FIG. 11C illustrate an auxiliary compressive support structure for a stent in various states of deployment with a stent; and

FIG. 12 illustrates a stent deployed on top of another stent.

While the disclosure is amenable to various modifications and alternative forms, specifics thereof have been shown by way of example in the drawings and will be described in detail. It should be understood, however, that the intention is not to limit the invention to the embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification.

All numeric values are herein assumed to be modified by the term “about,” whether explicitly indicated or not. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more features, structures, and/or characteristics. However, such recitations do not necessarily mean that all embodiments include the features, structures, and/or characteristics. Additionally, when features, structures, and/or characteristics are described in connection with one embodiment, such features, structures, and/or characteristics may also be used in connection with other embodiments whether explicitly described or not, unless clearly stated to the contrary.

In accordance with various principles of the present disclosure, an implantable device may be used to extend across an anatomical structure to control or regulate the size of a passage therethrough. For instance, an implantable device may extend across a body passage or lumen, such terms being used interchangeably herein without intent to limit. The body passage or lumen may include, without limitation, a portion of a passage or lumen, a passage or lumen between anatomical structures (passages, lumens, cavities, organs, etc.), a passage created across apposed tissue walls (such as to create an anastomosis) etc. The device has a passage or lumen (such terms being used interchangeably herein without intent to limit) therethrough which may be used to permit passage of material (e.g., fluid, liquid, chyme, food, etc.) between the anatomical structures in which the anastomosis is formed and in which the device is positioned. Thus, the device may be considered and referenced as an occlusion, lumen-apposing, or anastomosis or flow-enabling or flow-regulating or flow-controlling device, and such terms and various other alternatives thereto may be used interchangeably herein without intent to limit.

The following detailed description should be read with reference to the drawings in which similar elements in different drawings are numbered the same. The drawings, which are not necessarily to scale, depict illustrative embodiments and are not intended to limit the scope of the invention.

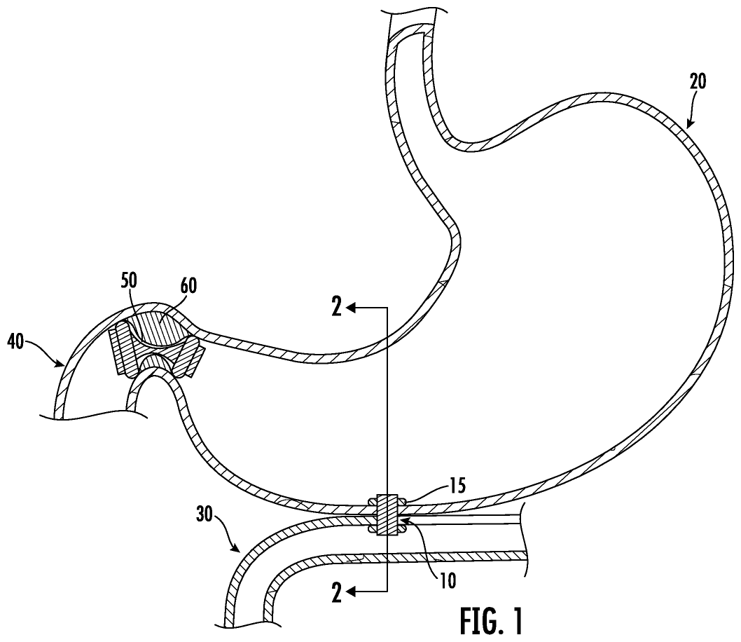

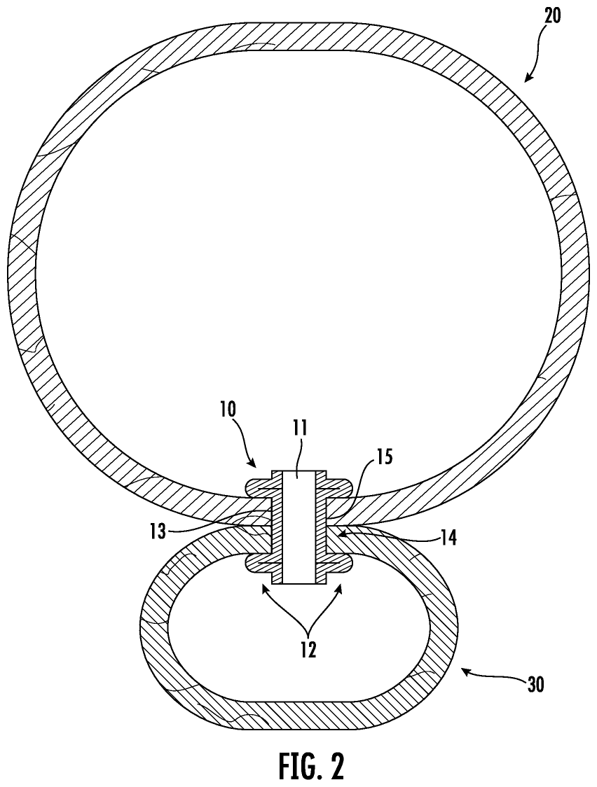

FIG. 1 illustrates a perspective view of an illustrative stent 10 positioned between a stomach 20 and a jejunum 30 (a portion of the small intestine), and FIG. 2 illustrates a cross-section view of the stent 10 positioned between the stomach 20 and the jejunum 30 taken at line 2-2 of FIG. 1. The stomach 20 normally passes food materials (e.g., chyme, partially digested food materials, fluids, etc.) into a duodenum 40 through a pylorus 60. In some cases, treatment for a patient experiencing obesity, diabetes, or duodenal ulcers may involve bypassing the duodenum 40, or restricting flow of materials through the duodenum 40. If the treatment requires complete bypass of the duodenum 40, then occlusion (e.g., full occlusion) of the pylorus 60 maybe indicated, and an anastomosis 15 maybe created between the stomach 20 and the jejunum 30, which may be known as a gastrojejunostomy. FIG. 1 illustrates an example bypass procedure in which a flow restricting device 50 has been positioned within the pylorus 60, thereby restricting access of food materials from the stomach 20 into the duodenum 40 (e.g., a complete bypass). A lumen-apposing metal stent (LAMS), such as the stent 10, may be placed between the stomach 20 and the jejunum 30, thereby forming the anastomosis 15, to allow for passage of food materials (fluid, liquid, chyme, etc.) from the stomach 20 and into the jejunum 30 through the lumen 11 of the stent 10, as shown in FIGS. 1 and 2.

The stent 10 is held in place by flanges 12 disposed on both proximal and distal ends of the stent 10. The flanges 12 are disposed on both ends of a medial region 13 which has a smaller radial diameter than the flanges 12. The flanges 12 and medial region 13 are configured to engage with a tissue surface 14 of both the stomach 20 and jejunum 30 and exert both a radial force and a compressive force upon the tissue 14 to aid retention of the stent 10 in the anastomosis 15.

While it is illustrated that the stent 10 maybe used in forming the anastomosis 15 between the stomach 20 and the jejunum 30, it may be contemplated that the stent 10 maybe used to drain a pancreatic fluid collection, pancreatic duct, bile duct, or gallbladder into the stomach or duodenum, used to treat a stenosis in a blood vessel, used to maintain a fluid opening or pathway in the vascular, urinary, biliary, tracheobronchial, esophageal or renal tracts, or position a device such as an artificial valve or filter within a body lumen, in some instances. Although illustrated as a stent, the stent 10 maybe any of several devices that may be introduced endoscopically, subcutaneously, percutaneously, or surgically to be positioned within an organ, tissue, or lumen, such as a heart, artery, vein, urethra, esophagus, trachea, bronchus, bile duct, pancreatic duct, pancreatic fluid collection, gallbladder or the like.

Once a lumen opposing stent is placed, it is critical that it stay in place, holding the two lumens together and bridging the openings in their walls. If it were to migrate out of place, the contents of the lumen could leak out into the peritoneal cavity. If partially digested food were to leak out of the stomach, if digestive juices were to leak out the bile or pancreatic duct, or if infected necrosis were to leak out a pancreatic fluid collection and into the abdomen, this would be a great risk to the health of the patient. The present disclosure provides methods, techniques, apparatuses, and systems to allow physicians to prevent migration of the lumen apposing stent.

The present disclosure provides devices that prevent migration of lumen apposing stents, and/or that anchor two tissue planes and/or a stent together, to prevent the distal tissue from moving away from the proximal tissue during and/or after stent placement.

In some embodiments of the disclosure, flange retention members are integrally disposed within the stent 10 and biased to exert a compressive force against the tissue 14 of the anastomosis 15. As noted, the stents described here can be used during several procedures, for example, when creating an anastomosis for gallbladder drainage.

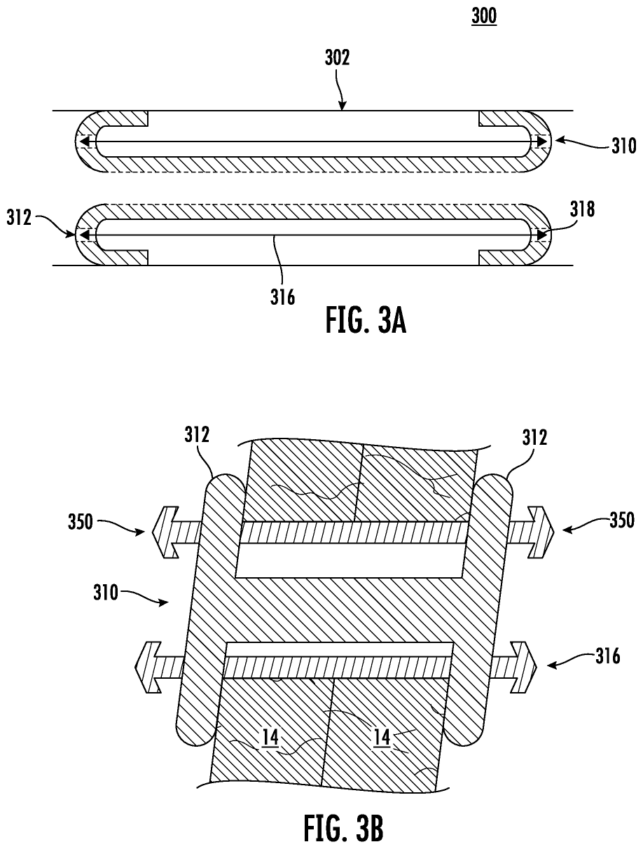

FIG. 3A and FIG. 3B illustrate a stent 310 comprising flange retention members 316. The flange retention members 316 include retention member ends 318 configured to engage with the flanges 312 of the stent 310. In some embodiment, retention member ends 318 can be hooks, arrows, or other mechanisms or shapes arranged to engage with and/or mechanically couple to the structure of stent 310. As a specific example, retention member ends 318 can be hook shaped and can hook over and/or mechanically couple to a portion of the wire mesh of both the distal and proximal flanges 312 of the stent 310. Further, as depicted a pair of flange retention members 316 is provided and integrally formed to couple to and exert compressive force against each pair of distal and proximal flanges 312.

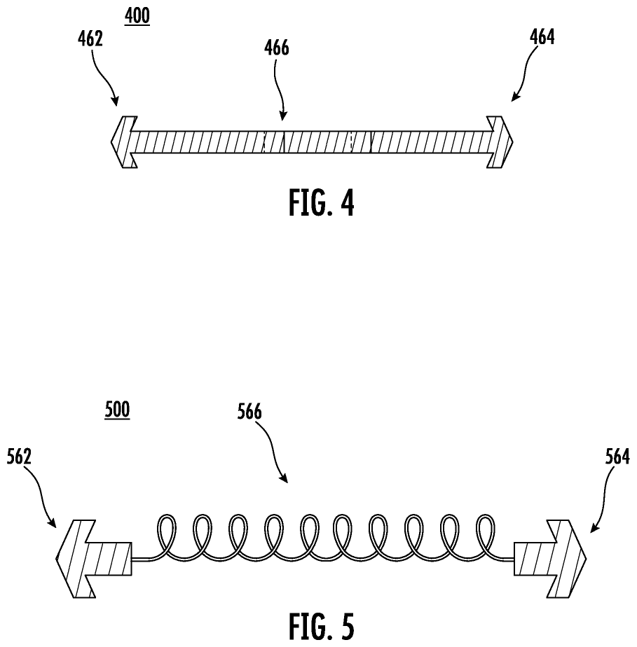

The flange retention members 316 may be made of nitinol and can include a variety of shapes or designs, for example, see FIG. 4 and FIG. 5. In general, the flange retention members 316 can be integrally packaged with the stent 310 and a delivery device. A variety of stent delivery devices are known in the art. For example, common stent delivery devices hold the stent 310 in a compressed extended state within a delivery sheath 302. The delivery sheath 302 is configured to be delivered to the site of the anastomosis via an endoscope. During delivery, the stent 310 is held in an extended flattened state by the sheath 302, as depicted in FIG. 3A. However, once deployed, the flange retention members 316 are biased to apply pressure in the direction of arrows 350 to pull the flanges 312 against the tissue 14 of the anatomical structures in which the anastomosis is formed (e.g., stomach 20 and jejunum 30, or the like).

It is important to note that although only two flange retention members 316 are depicted herein, a stent 310 could be provide with more than 2 flange retention members 316, for example, 3, 4, 5, 6, etc.

FIG. 4 illustrates a flange retention members 400, which can be implemented as flange retention members 316 in some embodiments. The flange retention member 400 includes a distal retention member end 462 and a proximal retention member end 464 as well as a central biased region 466. The central biased region 466 can comprise pleats or tubes that are folded or stacked within each other and biased to return to a shorted length when released from the delivery device sheath 302. Said differently, the central biased region 466 can be arranged to be compressed into an extended state and biased to return to a foreshortened state. As such, once deployed, the central biased region 466 may return the flange retention member 400 to the foreshortened state thereby, exerting pressure against flanges 312 in the direction of arrows 350.

FIG. 5 illustrates a flange retention members 500, which can be implemented as flange retention members 316 in some embodiments. The flange retention member 500 includes a distal retention member end 562 and a proximal retention member end 564 as well as a central biased region 566. The central biased region 566 can comprise a spring. The spring can be arranged to be stretched into an extended state and biased to return to a foreshortened state. As such, once deployed, the central biased region 566 may return the flange retention member 500 to the foreshortened state, thereby exerting pressure against flanges 312 in the direction of arrows 350.

As noted, stents are often used for drainage of adherent structures (e.g., pancreatic fluid collections, etc.). However, stents can also be used with non-adherent structures (e.g., stomach, gallbladder, and jejunum). If the stent shifts or is removed too early, there is a risk of leakage which can be catastrophic to the patient and require surgical intervention.

The present disclosure provides stents comprising an additional member arranged to provide inner support of the stent. The additional member and the inner support can reduce the chance the stent will collapse or shift position.

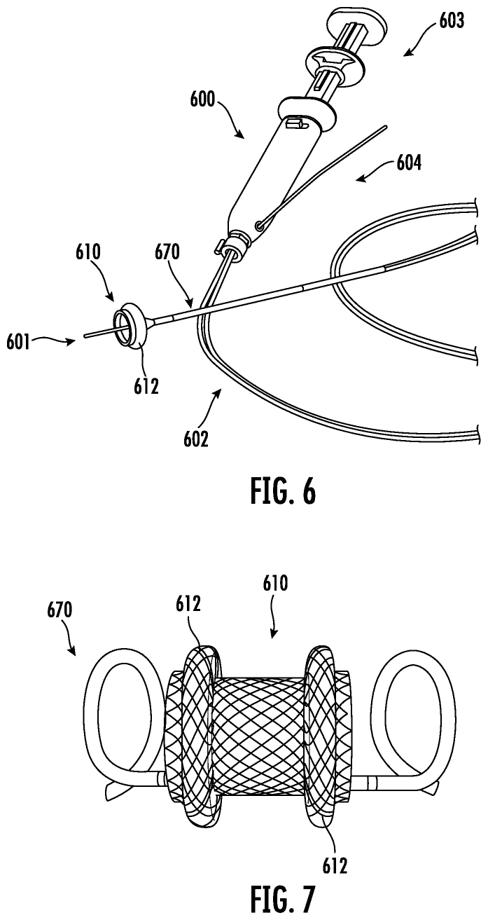

FIG. 6 illustrates a stent delivery device 600 having a handle 603 and a tip 601. The tip 601 can be configured to form an anastomosis 15 while the handle 603 can be configured to deploy the stent 610 into the anastomosis. The first flange (e.g., more distal flange) is deployed on the distal side of the anastomosis 15 and then the second flange (e.g., more proximal flange) is deployed on the proximal side of the anastomosis 15. As described above, delivery devices include a sheath in which the stent is retained in a compressed extended position. The delivery device 600 includes sheath 602 in which stent 610 can be deployed from. Further, delivery device 600 includes a plastic pigtail stent 670 mounted over or distal to the sheath 602 and a pigtail stent pusher 604 arranged to deploy the pigtail stent within the lumen of the stent 610 once the stent 610 is deployed (see FIG. 7). The pigtail stent pusher 670 can be used to push the pigtail stent 670 of the sheath such that the pigtail stent 670 deploys and the ends uncurl on both the proximal and distal sides of the stent 610.

The pigtail stent 670 sits within the lumen of the stent 610, and the two curled ends of the stent provide a secondary means of holding the two lumens together. The pigtail stent may be hollow, allowing for fluid flow and drainage, providing a backup if the primary lumen apposing stent 610 becomes clogged. In some embodiments, the delivery device 600 can be packaged to include both the stent 610 and the pigtail stent 670.

FIG. 7 illustrates an examples of a deployed stent 610 and pigtail stent 670. The outer diameter of the pigtail stent 670 may be small compared to the inner diameter of the stent 610. The length of the straight portion of the pigtail stent 670 and the length of stent 610 may be similar, so that they may both apply pressure to the lumen walls. It is to be appreciated that the devices shown in this figure are not to scale and further the size of the pigtail stent 670 relative to the stent 610 can vary.

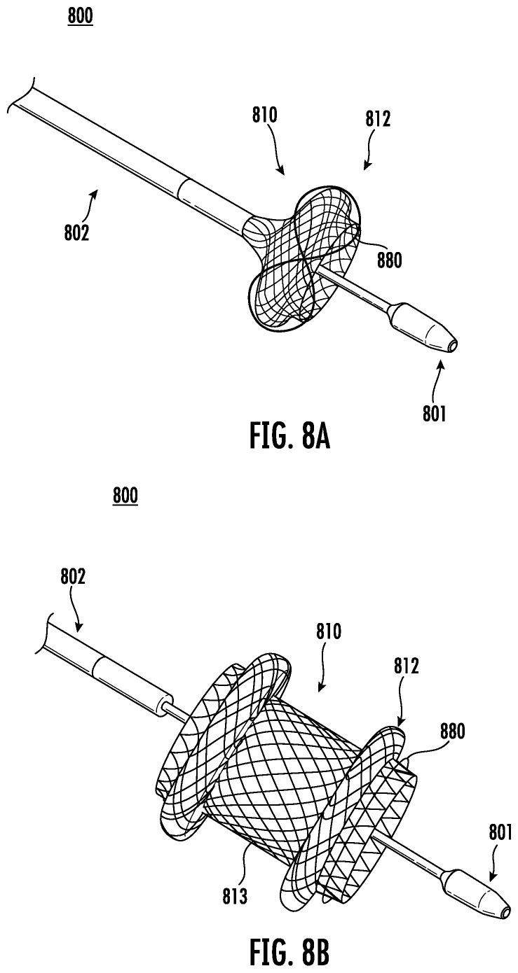

FIG. 8A and FIG. 8B illustrate a stent 810 and delivery system 800 including a sheath 802 in which the stent 810 is retained in a compressed and extended position and from which the stent 810 can be deployed. The delivery system 800 includes a tip 801 for forming an anastomosis as described above. The stent 810 further includes flanges 812 and a flange support structure 880. The auxiliary support structure 880 can be a shaped metal wire, spring, or other structure that when the stent 810 is deployed and the flange 812 opens to its expanded state, the auxiliary support structure 880 also opens and provides support to the flange 812. In some embodiments, one of the flanges can include a flange support structure 880. In other embodiments, both flanges 812 can include a flange support structure. In some embodiments, an auxiliary support structure 880 can be provides for the medial region 813 and addition to, or alternatively from, the flanges 812. Providing support to the flanges helps prevent migration of the stent out of the anastomosis.

With some embodiments, auxiliary support structure 880 can be provided in either or both the distal and proximal flanges 812, thereby the medial (or central saddle) region 813 can remain flexible while the flanges 812 are more rigid. As such, the radial and axial stiffness profiles of the flanges 812 can be decoupled from the medial region 813.

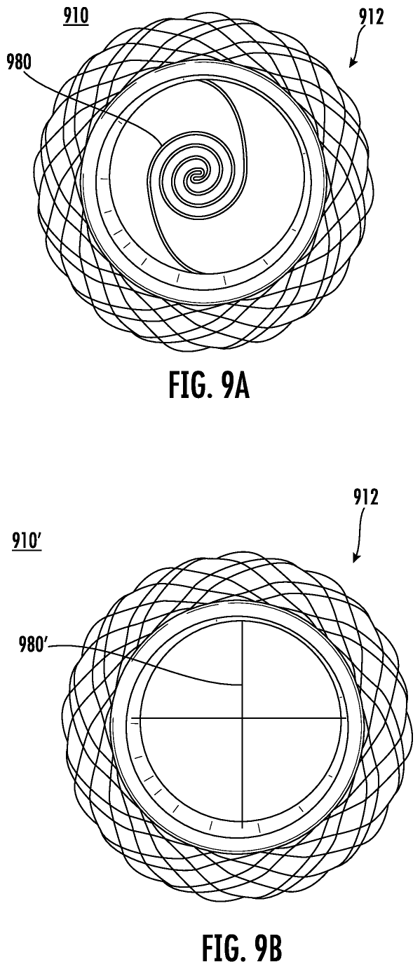

FIG. 9A and FIG. 9B illustrate alternatives to provide support to the flanges. FIG. 9A and FIG. 9B illustrate alternative designs of stent 910 and 910′, respectively, in the expanded state. As can be seen, the flange 912 as well as the auxiliary support structure 980 have expanded and the auxiliary support structure 980 provides radial and axial support to the expanded flange 912.

As noted above, stents are often placed between two non-adherent structures (e.g., as in a gastrojejunostomy, hepaticogastrostomy, gallbladder drainage into either the stomach or duodenum, or the like). Once an anastomosis is achieved, it is critical to assure that the stent will be maintained in place. For example, a migrated stent can lead to leakage of infectious material into the peritoneal cavity, resulting in infection or other complications and can require surgical intervention to correct. The present disclosure provides an auxiliary compressive and/or expansive support for a stent. In particular, the auxiliary compressive support structure can carry some of the tension between the two non-adherent structures thereby releasing some of the tension carried by the flanges of the stent.

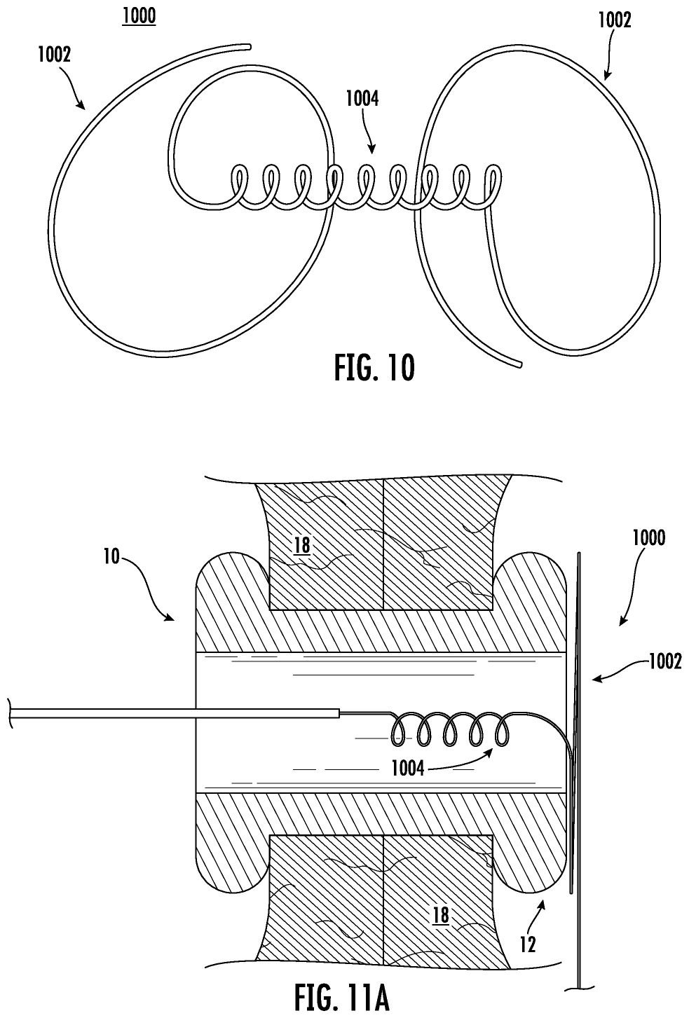

FIG. 10 illustrates an auxiliary compressive support structure 1000, which can be provided and deployed within a stent. The auxiliary compressive support structure 1000 comprises proximal and distal flange retention ends 1002 as well as a spring structure 1004 disposed between the retention ends 1002. The auxiliary compressive support structure 1000 can be formed form a metal or shape memory alloy (e.g., nitinol) and biased to return to the formed shaped. As such, the auxiliary compressive support structure 1000 can be flattened into a delivery device (e.g., catheter delivery tube, or the like) and delivered into the stent. The retention ends 1002 can be arranged (or shaped) to mechanically couple to the outside walls of flanges 12 of the stent 10. As such, the spring 1004 will release some tension from the flanges themselves and increase the force with which the stent 10.

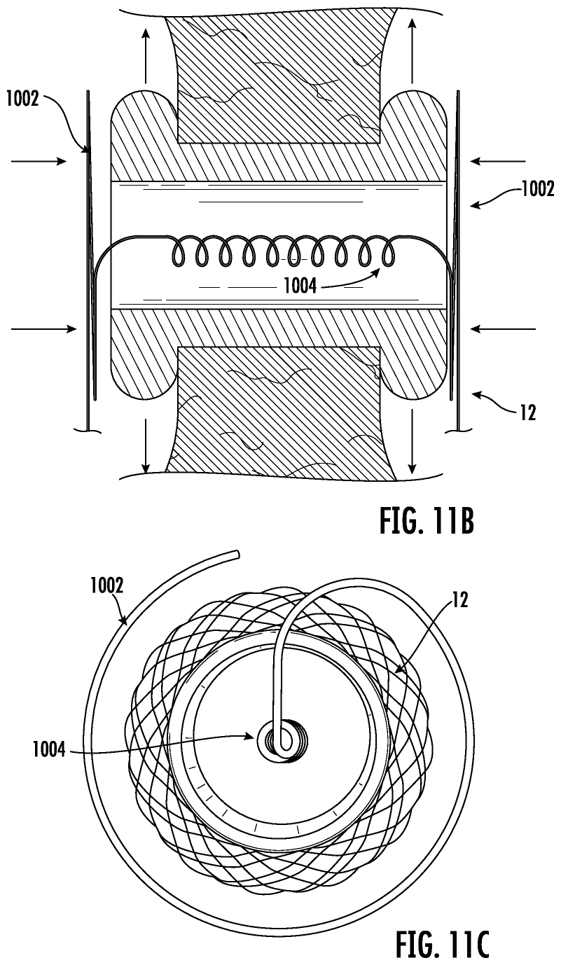

FIG. 11A, FIG. 11B, and FIG. 11C depict images showing a method of deploying the auxiliary compressive support structure 1000 of FIG. 10 into a deployed stent (e.g., like shown in FIG. 2. Turning first to FIG. 11A, once the stent 10 is deployed, the distal flange retention end 1002 of the auxiliary compressive support structure 1000 can be deployed (e.g., using a catheter and deployment handle, or the like) onto the distal flange 12 of the stent 10.

Turning to FIG. 11B, the proximal flange retention end 1002 of the auxiliary compressive support structure 1000 can be deployed (e.g., using the same catheter and deployment handle) onto the proximal flange 12 of the stent 10. It is to be appreciated that once both the distal and proximal flange retention end 1002 of the auxiliary compressive support structure 1000 are deployed onto the respective distal and proximal flanges 12, tension will be released from the flanges 12 of the stent 10 and further the stent 10 will be compressed more than it would be without the aid of the auxiliary compressive support structure 1000. As such, the diameter of the flanges 12 will increase. That is, the longitudinal length of the stent 10 will be reduced because of the compressive support of the auxiliary compressive support structure 1000, further resulting in the increase in the diameter of the flanges 12.

FIG. 11C illustrates an on axis view of a stent 10 with an auxiliary compressive support structure 1000 deployed therein. FIG. 11C shows a retention end 1002 coupled to a flange 12 and the spring 1004 disposed in the lumen of the stent 10. In some embodiments, the size and shape of the retention ends can be tailored for specific flange diameters. Further, the spring 1004 can be tailored to have a specific spring rate and unbiased length for various lengths of stents or procedures.

In another embodiment, the auxiliary compressive support structure 1000 maybe directly connected to the flanges of the stent 10 during manufacturing. Like the concept shown in FIG. 3, it may take on an elongated position when compressed into the delivery catheter, and take the shape shown in FIG. 11B once the stent 10 is deployed.

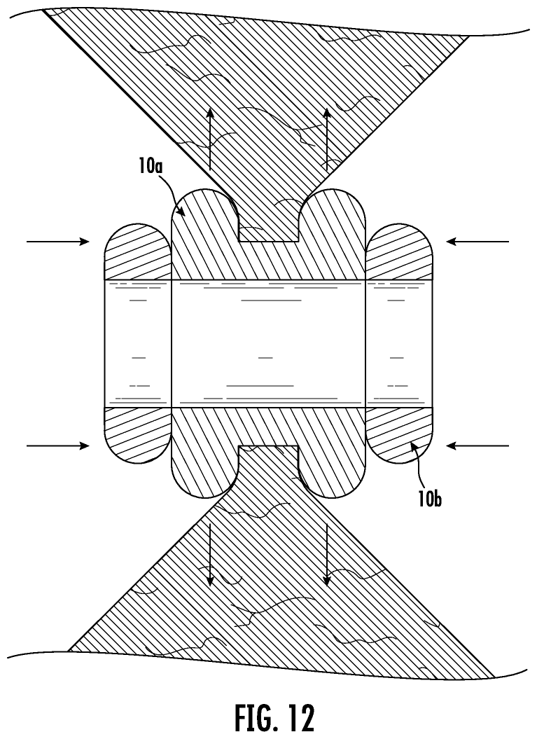

With some embodiments, an auxiliary compressive support structure 1000 can be provided by another stent. For example, FIG. 12 illustrates an inner stent 10a deployed in an anastomosis with an outer stent 10b deployed around the inner stent 10a. In some embodiments, the inner stent 10a may be provided with larger diameter flanges than the outer stent 10b while the outer stent 10b may be provided with a higher compressive force. In such a manner, the diameter of the flanges of the inner stent 10a can be increased as described above.

In general, the stents described herein can have an expanded outer diameter in the range of 6-70 millimeters (mm) and a foreshortened length (e.g., uncompressed length) in the range of 8-120 mm and a medial section diameter in the range of 5-30 mm. Further, the support structures described herein can have dimensions sized to mechanically couple to a deployed stent and provide the auxiliary support detailed herein.

In some embodiments, the devices described herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all the devices described herein may be doped with, made of, or otherwise include a radiopaque material. Radiopaque materials are understood to be materials capable of producing a relatively bright image on a fluoroscopy screen or another imaging technique during a medical procedure. This relatively bright image aids the user in determining the location of the devices during a procedure. Some example radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of devices.

In some embodiments, the materials may be compatible with Magnetic Resonance Imaging (MRI). Some materials that exhibit these characteristics include, for example, polymers, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, platinol, and the like, and others.

It is to be understood that this disclosure is, in many respects, only illustrative. Changes may be made in detail, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system, comprising:
a radially expanding tubular framework having a first end region, a second end region, a medial region positioned between the first end region and the second end region, and a lumen extending from the first end region to the second end region,
wherein the first end region comprises a first flange and the second end region comprises a second flange, and
an auxiliary support structure configured to provide support to the first flanges and the second flange when the stent is in an expanded state.

2. The system of claim 1, wherein the first flange and the second flange are configured to atraumatically engage a bodily tissue.

3. The system of claim 1, wherein the auxiliary support structure comprises a second radially expanding tubular framework, the second radially expanding tubular framework having a first end region, a second end region, a medial region positioned between the first end region and the second end region, and a lumen extending from the first end region to the second end region,
wherein the first end region of the second radially expanding tubular framework comprises a first flange and the second end region of the second radially expanding tubular framework comprises a second flange.

4. The system of claim 3, wherein the first flange and the second flange of the first radially expanding tubular framework have a first diameter and the first flange and the second flange of the second radially expanding tubular framework have a second diameter less than or equal to the first diameter.

5. The system of claim 4, wherein the first radially expanding tubular framework has a first longitudinal compressive force and the second radially expanding tubular framework has a second longitudinal compressive force greater than the first longitudinal compressive force.

6. The system of claim 1, wherein the auxiliary support structure comprises a proximal flange retention member, a distal flange retention member, and a spring coupled between the proximal and distal flange retention members.

7. The system of claim 6, wherein the first flange and the second flange of the radially expanding tubular framework have a first diameter and wherein the proximal and distal flange retention members comprise a second diameter, wherein the second diameter is greater than or equal to the first diameter minus 5 millimeters (mm) and less than or equal to the first diameter plus 5 mm.

8. The system of claim 6, wherein the spring has an unstretched length less than or equal to the foreshortened length of the stent.

9. A stent comprising:
a radially expanding tubular framework having a first end region, a second end region, a medial region positioned between the first end region and the second end region, and a lumen extending from the first end region to the second end region,
wherein the first end region comprises a first flange and the second end region comprises a second flange, and
at least one spring integrally coupled with the first flange and the second flange, the at least one spring biased to apply compressive force upon the flanges when the stent is in the foreshortened state.

10. The system of claim 9, wherein the spring comprises pleats or folds biased to apply compressive force on the first flange and the second flange.

11. The system of claim 9, wherein the spring comprises coils.

12. The system of claim 9, wherein the auxiliary support structure applies a compressive force onto the stent reducing the longitudinal length of the stent and increasing or maintaining the radial diameter of the first flange and the second flange.

13. The system of claim 9, wherein the stent comprises a coating.

14. The stent of claim 9, wherein the stent and/or the auxiliary support structure comprises nitinol.

15. The stent of claim 9, wherein the radially expanding tubular framework includes a coating applied over the radially expanding tubular framework.

16. A method comprising:
forming an anastomosis in tissue;
deploying a radially expanding tubular framework into the anastomosis, the radially expanding tubular framework comprising a first end region, a second end region, a medial region positioned between the first end region and the second end region, and a lumen extending from the first end region to the second end region,
wherein the first end region comprises a first flange and the second end region comprises a second flange, and
deploying an auxiliary support structure around the deployed radially expanding tubular framework.

17. The method of claim 16, wherein the auxiliary support structure comprises a second radially expanding tubular framework, the second radially expanding tubular framework having a first end region, a second end region, a medial region positioned between the first end region and the second end region, and a lumen extending from the first end region to the second end region, and wherein the first end region of the second radially expanding tubular framework comprises a first flange and the second end region of the second radially expanding tubular framework comprises a second flange.

18. The method of claim 17, wherein the first flange and the second flange of the first radially expanding tubular framework have a first diameter and the first flange and the second flange of the second radially expanding tubular framework have a second diameter less than or equal to the first diameter.

19. The method of claim 18, wherein the first radially expanding tubular framework has a first longitudinal compressive force and the second radially expanding tubular framework has a second longitudinal compressive force greater than the first longitudinal compressive force.

20. The method of claim 17, wherein the auxiliary support structure comprises a proximal flange retention member, a distal flange retention member, and a spring coupled between the proximal and distal flange retention members.
